(19) United States
(12) Patent Application Publication
(10) Pub. No.: US 2026/0215993 A1
(43) Pub. Date: Jul. 30, 2026

(54) LOUNGER FOR HYDROTHERAPY

(71) Applicant: KOHLER CO., Kohler, WI (US)

(72) Inventors: Jason KWACZ, Kohler, WI (US); Aurelian FURCOIU, Kohler, WI (US); Bhushan PATIL, Kohler, WI (US)

(73) Assignee: KOHLER CO., Kohler, WI (US)

(21) Appl. No.: 19/453,637

(22) Filed: Jan. 20, 2026

Related U.S. Application Data
(60) Provisional application No. 63/749,422, filed on Jan. 24, 2025.

(51) Int. Cl. A61H 9/00; A61F 7/00; A61F 7/02

(52) U.S. Cl. CPC A61H 9/0021; A61F 7/0053; A61F 7/007; A61F 7/0085; A61F 7/02; A61F 2007/0029; A61F 2007/0039; A61F 2007/0054; A61F 2007/0075; A61F 2007/0282; A61H 2009/0042; A61H 2201/0149; A61H 2201/0207; A61H 2201/0214; A61H 2201/0285; A61H 2201/1238; A61H 2201/1633; A61H 2201/1638; A61H 2201/1642; A61H 2201/5071; A61H 2201/5082; A61H 2205/06; A61H 2205/10

(57) ABSTRACT

This present disclosure is generally towards a lounger for hydrotherapy. The lounger can include a body seat, a plurality of sleeves, and a control unit. At least one of the sleeves is coupled to the body seat. At least one of the sleeves can include an air system including an air pump and an air bag and a water system including a water pump and a water bag. The air pump is configured to deliver air to the air bag and the water pump is configured to deliver water to the water bag. The control unit is communicably coupled to the air system and the water system to control operation of the lounger.

### CROSS-REFERENCE TO RELATED APPLICATION

The present application claims priority to U.S. Provisional Patent App. No. 63/749,422 filed on January 24, 2025, the disclosure of which is incorporated herein by reference in its entirety for all purposes.

### BACKGROUND

The present disclosure relates generally to a lounger. More specifically, the present disclosure relates to a lounger that can provide hydrotherapy to a user.

Hydrotherapy has a variety of benefits including improvement of sleep quality and mood, relief of muscles and joints, reduction of bloating and puffiness, and improvement in skin. Both cold and hot hydrotherapy can lead to improvement of a user. It would be advantageous for a system to provide one or more of these benefits.

### SUMMARY

At least one aspect of the present disclosure relates to a lounger for hydrotherapy. The lounger includes a body seat, a plurality of sleeves, and a control unit. At least one of the sleeves is coupled to the body seat. At least one of the sleeves or the body seat includes an air system including an air pump and an air bag and a water system including a water pump and a water bag. The air pump is configured to deliver air to the air bag and the water pump is configured to deliver water to the water bag. The control unit is communicably coupled to the air system and the water system to control operation of the lounger.

At least one aspect of the present disclosure relates to a lounger for hydrotherapy. The lounger a base and a lounger section coupled to the base. The lounger section includes an air system including an air bag configured to receive air at a pressure and a water system including a water bag coupled to the air bag. The water bag is configured to receive water at a temperature. The lounger section is configured to fit at least partially around a part of a user to deliver a hydrotherapeutic output from the air bag and the water bag to the part of the user.

At least one aspect of the present disclosure relates to a lounger section for a lounger. The lounger section includes a frame support coupled to a base of the lounger, an air system coupled to the frame support, and a water system coupled to the air system. The air system includes an air bag configured to contain air at a pressure, an air pump coupled to the air bag and configured to deliver air to the air bag, and an air pressure sensor configured to detect the pressure of air. The water system includes a water bag configured to contain water at a temperature, a water pump coupled to the water bag and configured to deliver water to the water bag, and a water temperature sensor configured to detect the temperature of water. The water bag is coupled to the air bag. The water temperature sensor and the air pressure sensor are communicably coupled to a control unit of the lounger.

### BRIEF DESCRIPTION OF THE DRAWINGS

FIG. 1 is a perspective view of a lounger, according to an exemplary embodiment.

FIG. 2 is an exploded view of the lounger of FIG. 1, according to an exemplary embodiment.

FIG. 3 is a perspective view of the lounger of FIG. 1, according to an exemplary embodiment.

FIG. 4 is a diagram of a body seat or a sleeve of the lounger of FIG. 1, according to an exemplary embodiment.

FIG. 5 is a perspective view of a sleeve of the lounger of FIG. 1, according to an exemplary embodiment.

FIG. 6 is an exploded view of the sleeve of FIG. 5, according to an exemplary embodiment.

FIG. 7 is a perspective view of a sleeve of the lounger of FIG. 1, according to an exemplary embodiment.

FIG. 8A a perspective view of a portion of a sleeve of the lounger of FIG. 1, according to an exemplary embodiment.

FIG. 8B is a perspective view of a portion of a sleeve of the lounger of FIG. 1, according to an exemplary embodiment.

FIG. 9 is a perspective view of a portion of a sleeve of the lounger of FIG. 1, according to an exemplary embodiment.

FIG. 10 is an exploded view of a portion of a sleeve of the lounger of FIG. 1, according to an exemplary embodiment.

FIG. 11 is a perspective view of a portion of the lounger of FIG. 1, according to an exemplary embodiment.

FIG. 12 is a perspective view of a portion of the lounger of FIG. 1, according to an exemplary embodiment.

FIG. 13 is a perspective view of a water system of the lounger of FIG. 1, according to an exemplary embodiment.

FIG. 14 is a perspective view of a portion of the water system of FIG. 13, according to an exemplary embodiment.

FIG. 15 is a control diagram of the lounger of FIG. 1, according to an exemplary embodiment.

### DETAILED DESCRIPTION

Referring generally to the FIGURES, disclosed herein is a lounger for hydrotherapy. The lounger can provide hydrotherapy to a user by using both hot therapy and cold therapy through water. The lounger can include a body seat and/or a sleeve to fully enclose a user of the lounger. The lounger includes a water system and an air system in the body seat and the sleeve to circulate water at different pressures to provide the user with a massage for example. A temperature of the water can be adjusted (e.g., hot, cold) through the water system to transport water to a water bag. The water bag provides pressure on a user through adjustment of an air pressure of the air system that presses against the water bag. Additionally, the lounger can provide targeted hydrotherapy by using sensors to determine areas of the user that needs hydrotherapy and delivering the necessary hydrotherapy (e.g., water temperature, pressure) to the determined area. The user can also control the lounger through use of a user interface.

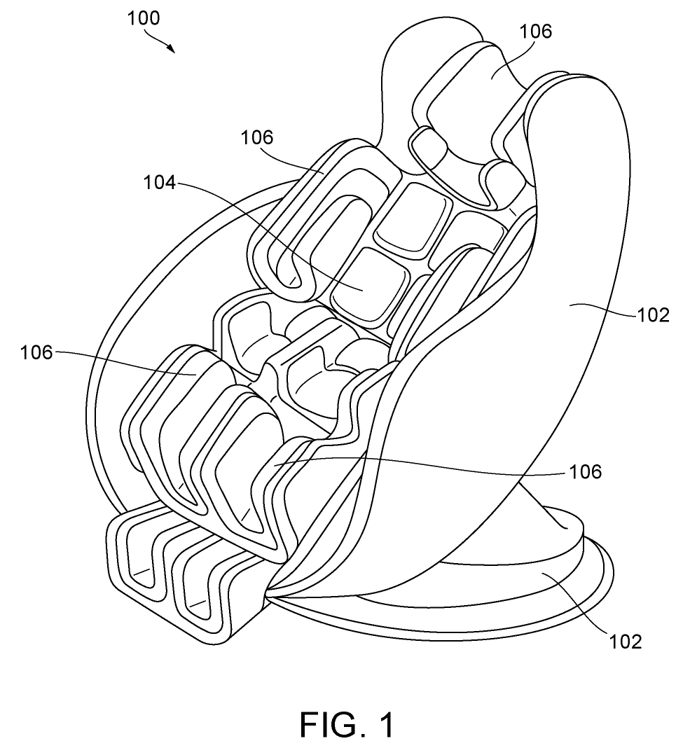

FIG. 1 depicts a lounger 100, according to an exemplary embodiment. The lounger 100 is configured to provide a number of hydrotherapeutic outputs to a user of the lounger 100. The lounger 100 can receive and release water and/or air to provide treatment to the user. As shown in FIG. 1, the lounger 100 includes a base 102. The base 102 may be a frame and is an outer structure of the lounger 100. The base 102 may provide a chair-like structure of the lounger 100, upon which a plurality of lounger sections 103 may be supported. The base 102 is configured to provide a support for the lounger 100. The base 102 may be manufactured from metal (e.g., steel, aluminum) or plastic, among other materials. In various embodiments, at least a portion of the base 102 is positioned on a ground (e.g., a floor). For example, the base 102 may be coupled to the ground. In various embodiments, an aperture is used to couple the base 102 to the ground.

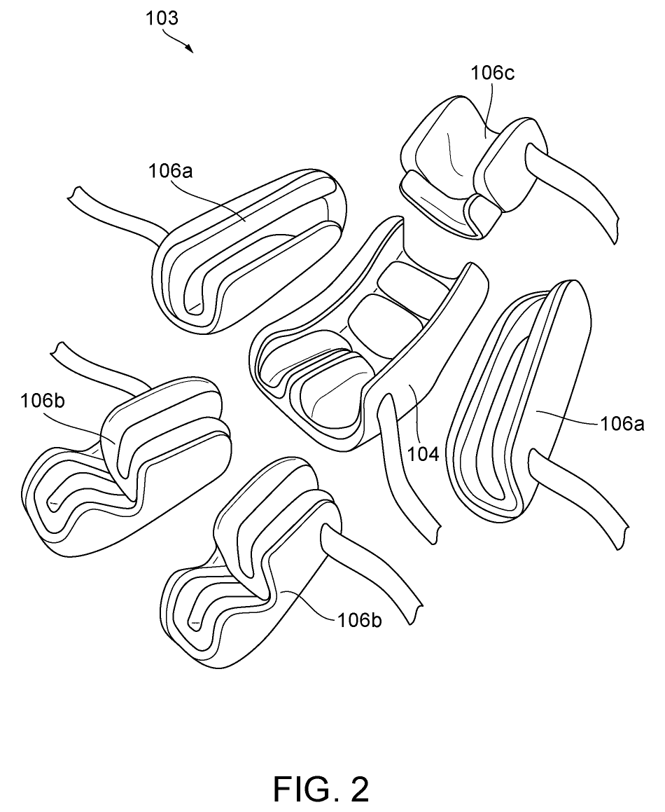

As shown in FIG. 2, the lounger 100 includes the plurality of lounger sections 103 (e.g., cushions, seats, sleeves, etc.). The plurality of lounger sections 103 are configured to receive and release water and/or air. At least a portion of the user of the lounger 100 may engage with a lounger section 103. The lounger section 103 is configured to fit at least partially around a part of the user to deliver hydrotherapeutic outputs from the lounger section 103 to the user. For example, the user may receive pressure from the lounger section 103 at a temperature dependent on the water temperature of the water in the lounger section 103. In various embodiments, at least one of the plurality of lounger sections 103 may be a body seat (e.g., body seat 104). As shown in FIG. 1, the lounger 100 includes a body seat 104. At least a portion of the body seat 104 is coupled to the base 102. The body seat 104 is configured to support a portion of the user of the lounger 100. For example, the user of the lounger 100 can place at least a portion of a body (e.g., a torso) of the user on the body seat 104.

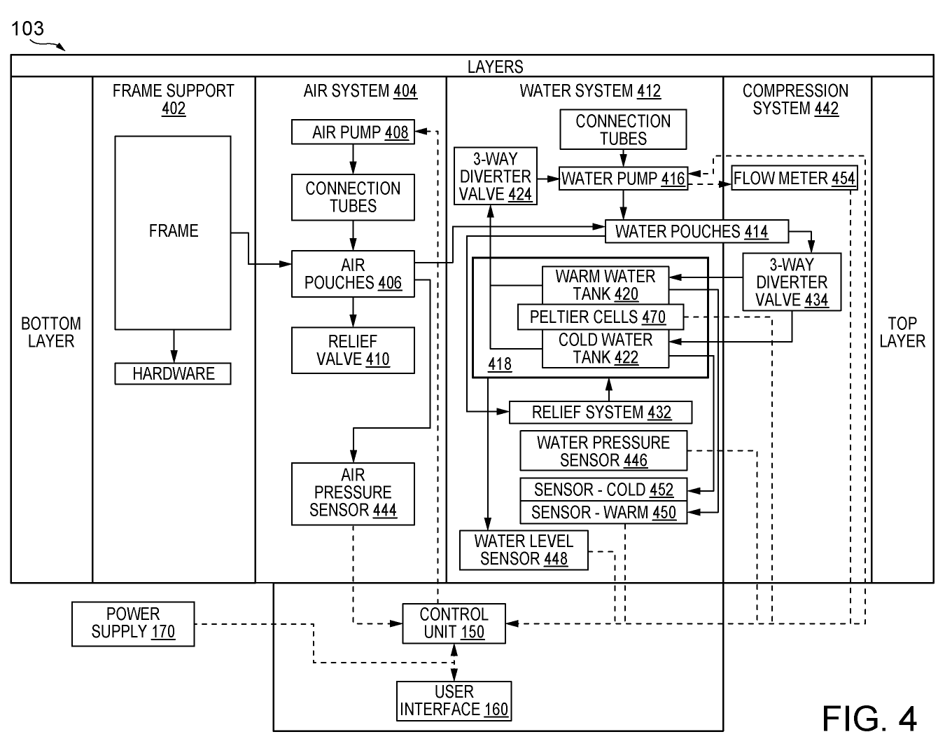

As shown in FIG. 4, at least one of the plurality of lounger sections 103 (e.g., the body seat 104) may have a number of layers to provide hydrotherapeutic outputs. In various embodiments, the layers are stacked on top or adjacent each other. In other embodiments, although shown as distinct layers, the layers are overlapped, such that one layer may be combined within the same layer as another layer. Although described in context with the body seat 104, the layers shown in FIG. 4 may be applicable to others of the plurality of lounger sections 103 (e.g., sleeve 106).

As shown in FIG. 4, the lounger sections 103 (e.g., body seat 104) can have a frame support 402 to support the remaining layers and the user. The frame support 402 may be coupled to the base 102, such that the frame support 402 is a bottom layer, or the layer adjacent the base 102, of the body seat 104. The frame support 402 may have a structure that substantially matches and/or conforms to a portion of a user, such as a human body part, where the lounger section 103 (e.g., the body seat 104) is configured to support and/or fit. For example, the frame support 402 for the body seat 104 may have a structure and/or a shape similar to a torso of a human. The frame support 402 can be a solid frame manufactured from a plastic or a metal (e.g., steel, aluminum). The frame support 402 can include connection points for hardware needed to deliver hydrotherapeutic outputs to the user, as described herein.

The lounger sections 103 (e.g., body seat 104) can include an air system 404. The air system 404 may be coupled to the frame support 402. The air system 404 is configured to provide air to the body seat 104 for therapy usage of the lounger 100. The air system 404 includes at least one air bag 406. The air bag 406 can be a pouch or a container and is configured to contain air to provide pressure to the user of the lounger 100. The air bag 406 can be coupled to the frame support 402. In various embodiments, the air system 404 includes a number of air bags 406 coupled around the frame support 402 to provide pressure to various parts of the user through the deliverance of air. The air system 404 also includes an air pump 408 coupled to the air bag 406. The air pump 408 can be coupled to the air bag 406 by connection tubes. The air pump 408 is used to deliver (e.g., pump) air into the air bag 406. Air delivered to the air bag 406 by the air pump 408 can be from an environment (e.g., an atmosphere). In other embodiments, the air may be delivered from an air supply (e.g., an air tank). The air pump 408 can be manual or electric. For example, as described herein, the air pump 408 can be communicatively coupled to a control unit (e.g., control unit 150) to provide air to the air bag 406.

The air system 404 includes a relief valve 410. The relief valve 410 can release air from the air bag 406. For example, the relief valve 410 may release air when the user is done using the lounger 100. In another example, the relief valve 410 may release air to adjust a pressure of the air bag 406 and change the pressure received by the user. In such example, the relief valve 410 may release air when a pressure of the air bag 406 is detected (e.g., by a control unit, as described herein) to be too high. Air released by the relief valve 410 can be released into an environment (e.g., an atmosphere). In another embodiment, air may be released by the relief valve 410 into an air supply (e.g., an air tank).

The lounger sections 103 (e.g., body seat 104) can include a water system 412. The water system 412 may be coupled to the air system 404. In various embodiments, the water system 412 is integrated with the air system 404. The water system 412 is configured to provide water to the body seat 104 for hydrotherapy usage of the lounger 100. The water system 412 may be the layer of the body seat 104 proximate the user when the user is using the lounger 100 such that at least a portion of the water system 412 is contacting the user during use. The water system 412 includes at least one water bag 414. The water bag 414 is configured to receive water. The water bag 414 may be manufactured from a flexible material allowing the water bag 414 to change in shape. As a result, the water bag 414 may be conformed to a shape of the user (e.g., to fit around at least a portion of the user). The water bag 414 may be proximal to the user such that a skin of the user is contacting the water bag 414 when the lounger 100 is in use. In various embodiments, the water bag 414 is coupled to the air bag 406. For example, air received into the air bag 406 can cause the air bag 406 to inflate and the inflated air bag 406 can generate pressure to push against the water bag 414 such that the water bag 414 is pushing against the user to provide hydrotherapy. The water bag 414 can be coupled to the air bag 406 by adhesive, tape, stitching, ultrasound welding, or fasteners (e.g., hook-and-loop fasteners), among others.

The water system 412 can include a water pump 416 coupled to the water bag 414 to provide water to the water bag 414. In various embodiments, the water pump 416 is coupled to the water bag 414 by connection tubes. The water pump 416 may electrically deliver water to the water bag 414. For example, the water pump can be communicatively coupled to a control unit (e.g., control unit 150) to provide water to the water bag 414.

The water pump 416 can receive the water from a water tank system 418. The water tank system 418 is configured to store water and deliver water from the water tank system 418 (e.g., to the water pump 416) for the water bag 414. In various embodiments, the water tank system 418 is a part of the water system 412 (and the lounger 100). In other embodiments, the water tank system 418 is positioned external at least one of the water system 412 or the lounger 100. In various embodiments, the water tank system 418 is configured to deliver water to more than one water pump 416 and/or more than one lounger sections 103. For example, the water tank system 418 may be configured to deliver water to both a water pump 416 for the body seat 104 and to a water pump 416 for the sleeve 106.

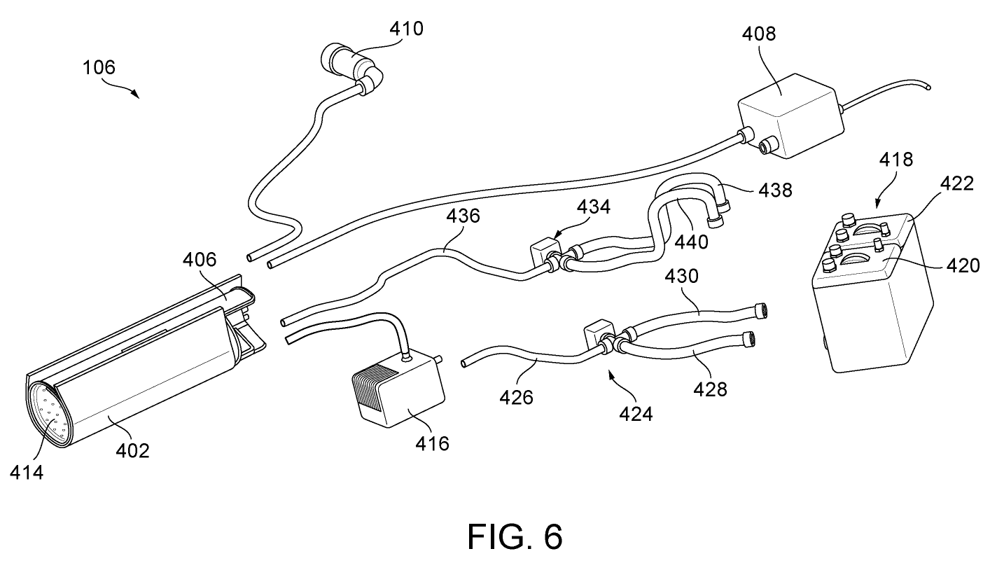

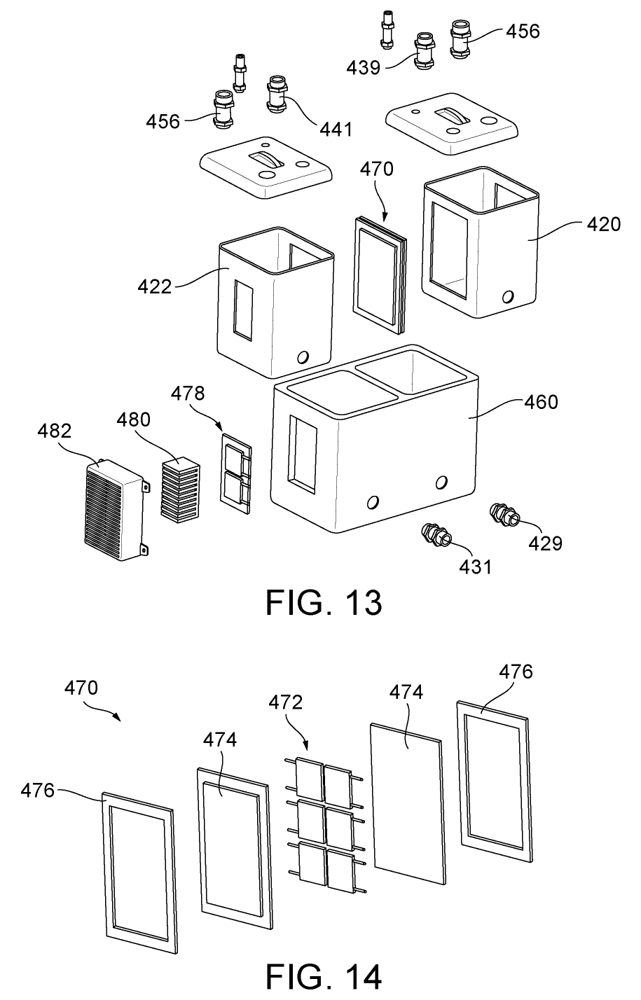

The water tank system 418 can include a first tank (e.g., hot water tank 420) and a second tank (e.g., cold water tank 422). The hot water tank 420 and the cold water tank 422 can be positioned within a tank cover 460, as depicted in FIGS. 6 and 13. The hot water tank 420 and the cold water tank 422 are configured to hold water. For example, the hot water tank 420 can hold water at a first temperature and the cold water tank 422 can hold water at a second temperature. In various embodiments, the first temperature is higher than the second temperature, such that the hot water tank 420 contains hot water and the cold water tank 422 contains cold water. The hot water tank 420 can provide the hot water to the water pump 416 and the cold water tank 422 can provide the cold water to the water pump 416. In various embodiments, the water tank system 418 includes only one tank. In such embodiments, the one tank may be configured to store and deliver both cold water and hot water. The one tank may have a heating/cooling assembly integrated within the tank to adjust the temperature of the water as needed.

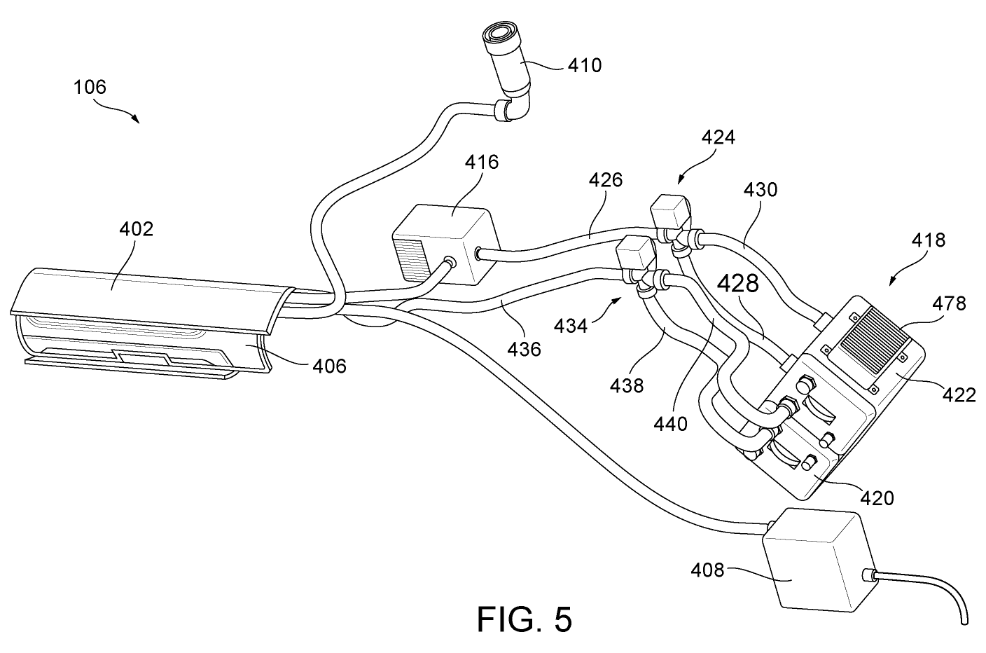

As shown in FIGS. 5-6, among others, the water pump 416 can be coupled to the water tank system 418 by a first three-way diverter valve 424. The first three-way diverter valve 424 can have a first member 426 that connects to the water pump 416 to provide the water from the water tank system 418. The first three-way diverter valve 424 can have a second member 428 that connects to the hot water tank 420 by a member connector 429. The second member 428 can direct the hot water from the hot water tank 420 to the first member 426 and the water pump 416. The first three-way diverter valve 424 can include a third member 430 that connects to the cold water tank 422 by a member connector 431. The third member 430 can direct the cold water from the cold water tank 422 to the first member 426 and the water pump 416 for delivery to the water bag 414. As such, either hot water and cold water can be provided to the water bag 414 by the water pump 416 and the first three-way diverter valve 424 to provide the user with both hot water and cold water hydrotherapy, as necessary.

In various embodiments, the water tank system 418 can include at least one Peltier assembly 470 to keep the water in hot water tank 420 hot and the water in the cold water tank 422 cold. As shown in FIG. 14, the Peltier assembly 470 can include at least one Peltier module plate 472 and is manufactured from a semiconductor material. The Peltier module plate 472 has a first side that absorbs heat for cooling and a second side that releases heat for heating. The first side is directed towards the cold water tank 422 and the second side is directed towards the hot water tank 420. The Peltier module plate 472 is positioned between a first aluminum plate 474 and a second aluminum plate 474 to provide structural support. Although aluminum is used for the first and the second aluminum plate 474, it can be appreciated by those skilled in the art that other materials (e.g., other ceramics) can be used for the first and the second aluminum plate 474. A gasket (e.g., a silicon rubber gasket 476) is coupled to the first and the second aluminum plate 474. As such, the Peltier assembly 470 is coupled to the hot water tank 420 by the silicon rubber gasket 476 on a first side of the Peltier assembly 470 and is coupled to the cold water tank 422 by the silicon rubber gasket 476 on a second side of the Peltier assembly 470.

As shown in FIG. 13, in various embodiments, the water tank system 418 includes a second Peltier module plate 478 that is coupled to the tank cover 460 and the cold water tank 422 for cooling water in the cold water tank 422. A first side of the second Peltier module plate 478 that absorbs heat for cooling is directed towards the cold water tank 422. A second side of the second Peltier module plate 478 that releases heat for heating is coupled to a heat sink 480. The heat sink 480 prevents overheating from the second side of the second Peltier module plate 478. In various embodiments, the water tank system 418 includes a heat sink cover 482 for the second Peltier module plate 478.

In various embodiments, a heat pump (e.g., a solid-state heat pump, a Stirling heat pump) can be used to heat water in the hot water tank 420 and cool water in the cold water tank 422. For example, the water tank system 418 may include a Stirling heat pump to compress and expand a gas (e.g., helium, nitrogen) to heat and cool the hot water tank 420 and the cold water tank 422, respectively. Additionally, the water tank system 418 may include a regenerator that stores and transfers heat to cool and heat as needed. The water tank system 418 can also include a piston to move the gas between the hot water tank 420 and the cold water tank 422 dependent on whether the gas is compressing or expanding.

As shown in FIG. 4, the water system 412 can include a relief system 432 to release water from the water bag 414. The water may be released back into the water tank system 418. For example, the relief system 432 can be a relief valve similar to the relief valve 410 for the air system 404. The relief system 432 may be configured to transport the water from the water bag 414 and into the water tank system 418.

In various embodiments, the relief system 432 includes a second three-way diverter valve 434 that connects the water bag 414 to the water tank system 418. Use of the second three-way diverter valve 434 allows for water to be returned to the water tank system 418 based on the temperature of the water. For example, hot water may be returned to the hot water tank 420 and cold water may be returned to the cold water tank 422. The second three-way diverter valve 434 can include a first member 436 that is coupled to the water bag 414 to transport the water in the water bag 414 to the water tank system 418. The second three-way diverter valve 434 can include a second member 438 connected to the hot water tank 420 by a return connector 439 to direct hot water from the water bag 414, through the first member 436, and to the hot water tank 420. The second three-way diverter valve 434 can include a third member 440 connected to the cold water tank 422 by a return connector 441 to direct cold water from the water bag 414. The cold water from the water bag 414 is directed through the first member 426, to the third member 440 and to the cold water tank 422.

As further described herein, in various embodiments, the lounger sections 103 (e.g., body seat 104) can include a compression system 442. The compression system 442 is configured to detect and determine an applied pressure from the water system 412 and the air system 404. The compression system 442 may be communicatively coupled to the control unit 150 to communicate information related to the applied pressure such that operation of the lounger sections 103 (e.g., the air system 404, the water system 412) can be adjusted. In various embodiments, as shown in FIG. 4, the compression system 442 includes the second three-way diverter valve 434.

In various embodiments, at least one of the plurality of lounger sections 103 may be a sleeve (e.g., sleeve 106). As shown in FIG. 1, the lounger 100 includes a number of sleeves 106. At least a portion of the sleeve 106 is coupled to the base 102. In various embodiments, a portion of the sleeves 106 is coupled to the body seat 104. A portion (e.g., a body part) of the user can be positioned (e.g., fitted) within the sleeves 106. In other words, as shown in FIG. 2, the sleeves 106 may be configured to fit to a body part of the user. For example, at least one of the sleeves 106 may be an arm sleeve 106a for the user to position an arm of the user within. In another example, at least one of the sleeves 106 may be a leg sleeve 106b for the user to position a leg of the user within. In yet another example, at least one of the sleeves 106 may be a head sleeve 106c conformed for a head of the user.

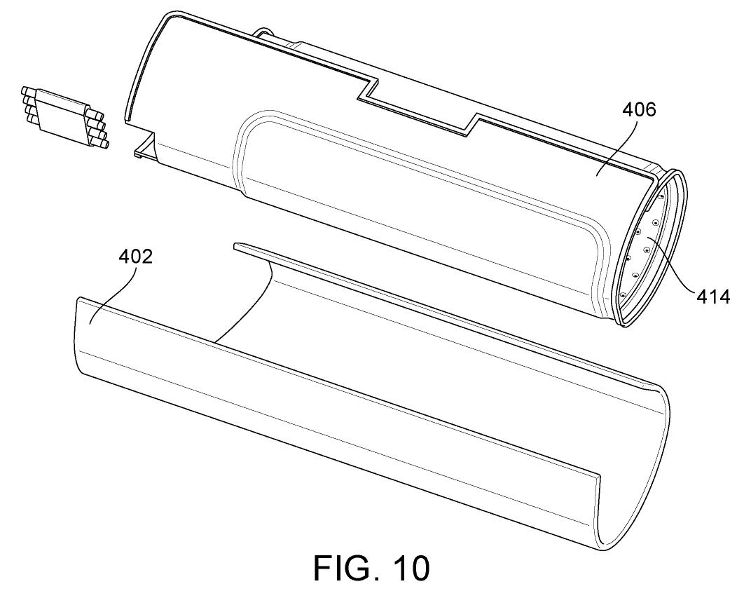

As described herein and shown in FIG. 4, at least one of the plurality of lounger sections 103 (e.g., the body seat 104, the sleeve 106) can include the layers (e.g., the frame support 402, the air system 404, the water system 412, the compression system 442) to provide hydrotherapeutic outputs. At least one of the sleeves 106 can include the frame support 402, the air system 404, the water system 412, and the compression system 442, as described herein. In various embodiments, the frame support 402, and therefore the air system 404 and the water system 412 can be conformed to a general shape of a portion of the user that the sleeve 106 is designed for. For example, the frame support 402 of the arm sleeve 106a, as shown in FIGS. 9 and 10, can have a curved shape (e.g., a U-shape) to closely fit to the arm of the user.

In various embodiments, as shown in FIG. 9, the frame support 402 of the arm sleeve 106a can include an upper arm frame 502 and a lower arm frame 504. The upper arm frame 502 can be configured to fit an upper arm (e.g., distal to a hand of the arm) of the arm of the user. The lower arm frame 504 can be configured to fit a lower arm (e.g., proximal to the hand) of the arm of the user. The upper arm frame 502 and the lower arm frame 504 can be coupled by a joint frame 506 that can fit around an elbow of the arm. The configuration of the frame support 402 with the upper arm frame 502, the lower arm frame 504, and the joint frame 506 can provide additional comfort and mobility to the user using the lounger 100. Similarly, the frame support 402 of the leg sleeve 106b can include an upper leg frame, a middle leg frame, a lower leg frame, or some combination thereof. The upper leg frame can be configured to fit an upper leg (e.g., distal to a feet of the leg) of the leg of the user. The middle leg frame can be configured to fit a lower leg (e.g., proximal to the feet) of the leg. The lower leg frame can be configured to fit the feet of the user. The upper leg frame and the middle leg frame can be coupled by a joint frame that can fit around a knee of the leg. The middle leg frame and the lower leg frame can be coupled by a joint frame that can fit around an ankle of the leg.

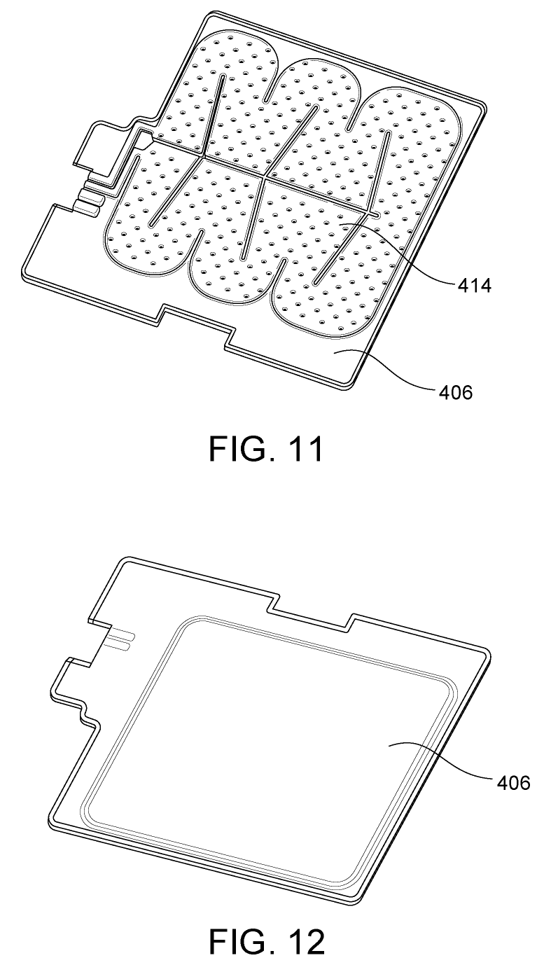

FIGS. 11 and 12 show a front view and a back view, respectively, of a portion of the lounger section 103, according to an example embodiment. As described herein, during operation of the lounger 100, the lounger section 103 (e.g., the air bag 406, the water bag 414) receives air and/or water. The received air and water and the pressure and temperature of the air and water provides hydrotherapeutic outputs to a user (e.g., a part of the user engaged with the lounger section 103. As described herein with reference to FIG. 4, in various embodiments, the air system 404 is positioned closer to a bottom layer and the water system 412 is positioned closer to a top layer of the lounger section 103. As a result, in various embodiments, the water bag 414 is directly engaged with the user during operation of the lounger 100. As shown in FIG. 11, the air bag 406 is coupled to the water bag 414. The received air by the air bag 406 and the pressure of the air may directly cause movement and pressure changes of the water bag 414 to provide pressure to the user at a temperature determined by the water temperature of the water bag 414. The positioning of the water bag 414 closer to the user may aid in providing thermal outputs from the water temperature in the water bag 414 to the user.

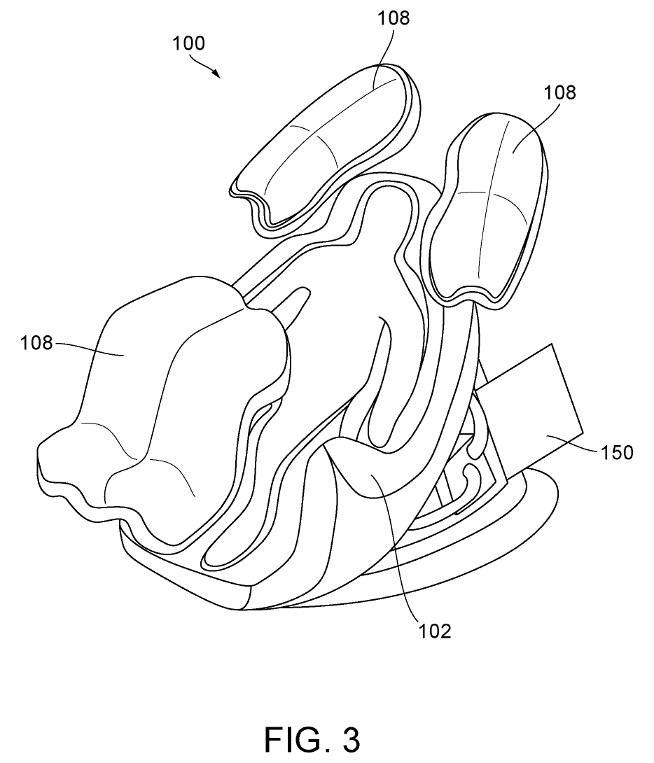

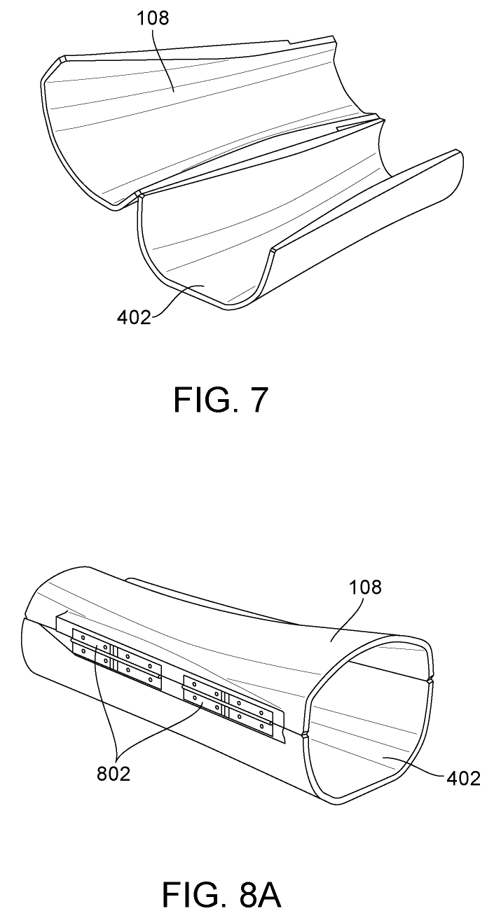

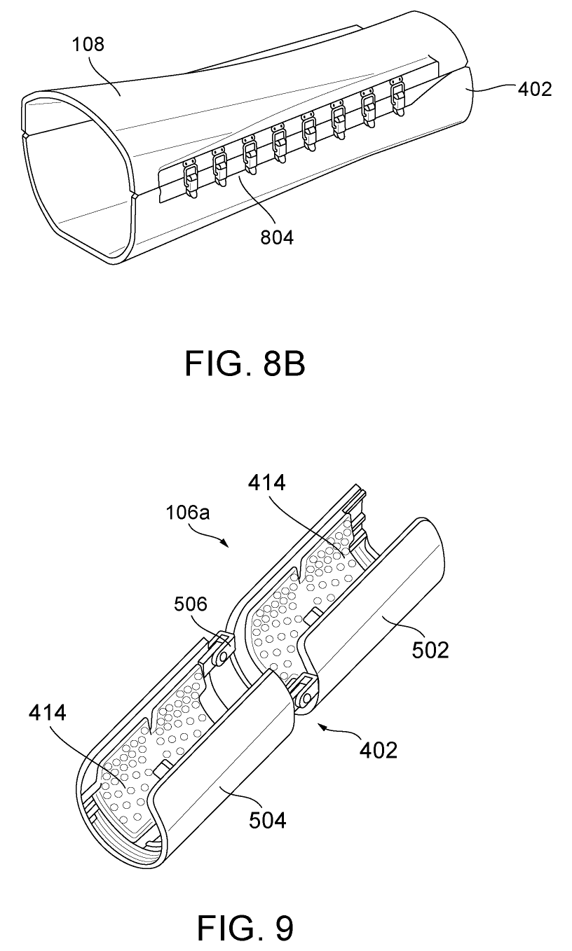

In various embodiments, as shown in FIGS. 3 and 7, the lounger 100 includes at least one top cover 108. The top cover 108 can be coupled to the sleeves 106 or the body seat 104. As described herein, the lounger section 103 (e.g., the sleeve 106, the body seat 104) may fit at least partially around a part of the user. The top cover 108 is configured to fully enclose the part of the user between the sleeves 106 and/or body seat 104 and the top cover 108. In other words, the top cover 108 and the lounger section 103 (e.g., the sleeve 106, the body seat 104) forms (e.g., defines) an enclosure. The enclosure can fit the portion (e.g., part) of the user. In various embodiments, the top cover 108 is of a same type as the body seat 104 and/or the sleeve 106. For example, the top cover 108 may include at least one of the layers as described with reference to FIG. 4 (e.g., the frame support 402, the air system 404, and the water system 412, the compression system 442). As shown in FIGS. 8A and 8B, the top cover 108 can be coupled to the sleeve 106 by at least one hinge 802 and at least one spring clamp 804. In various embodiments, fasteners (e.g., hook-and-loop fasteners) are used to couple the top cover 108 to the sleeve 106. Other coupling methods (e.g., tape, adhesives, glue, etc.) can be used to couple the top cover 108 to the sleeve 106 and fully enclose the user between the sleeve 106 or the body seat 104 and the top cover 108. In various embodiments, the top cover 108 is removably coupled from the lounger section 103 (e.g., the sleeve 106, the body seat 104).

In various embodiments, the lounger 100 does not include the top cover 108, and the portion of the user can be fully enclosed by the body seat 104 or the sleeve 106 using the air system 404 and/or the water system 412 of the body seat 104 or the sleeve 106. For example, as shown in FIG. 10, a first end of the air bag 406 can wrap around a portion of the user to couple to a second end of the air bag 406 and from an enclosure to enclose the portion of the user. The first end of the air bag 406 can couple to the second end of the air bag 406 with fasteners (e.g., hook-and-loop fasteners) or tape, among other coupling mechanisms.

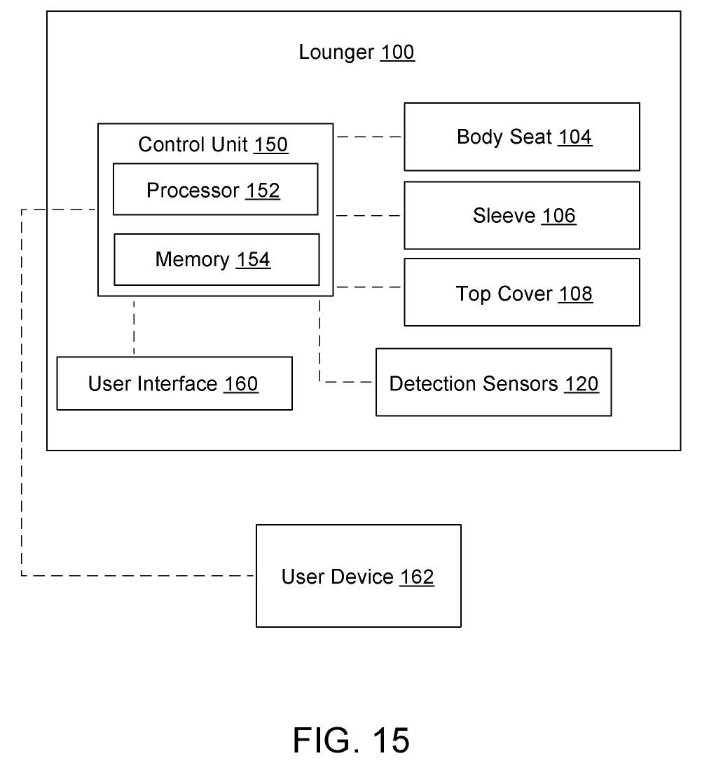

As shown in FIGS. 3 and 15, among others, the lounger 100 can also include a control unit 150 to control operation of the lounger 100. The control unit 150 can be coupled to the lounger sections 103 (e.g., the body seat 104 and/or the sleeves 106) to monitor hydrotherapeutic outputs and responses from the user and emit signals dependent on the monitored hydrotherapeutic outputs and responses from the user. The control unit 150 may be configured to interpret data given from a user or controls. For example, the control unit 150 may be configured to receive a user input related to a desired temperature of the hydrotherapeutic outputs from at least one of the lounger sections 103 and control the at least one of the lounger sections 103 (e.g., the water system 412) to deliver water at the desired temperature. The control unit 150 includes one or more processors 152 communicably coupled to memory 154. The one or more processors 152 is configured to process and control operations of given processes or data and the memory 154 stores data and information for operation of the lounger 100. For example, the control unit 150 may be used to program the air pump 408 of a lounger section 103 (e.g., the body seat 104, the sleeve 106) to increase an air pressure in the air bag 406, as described herein.

As shown in FIG. 4, the air system 404 can include an air pressure sensor 444 that is communicatively coupled to the control unit 150. The air pressure sensor 444 may be coupled to the air bag 406. The air pressure sensor 444 is configured to detect an air pressure of the air bag 406 and/or a change in the air pressure of the air bag 406. As described herein, the air pressure of the air bag 406 changes as the air pump 408 and the relief valve 410 add and remove air from the air bag 406, respectively. The air pressure sensor 444 can receive information related to the air pressure (e.g., determine and/or detect the air pressure). Information from the air pressure sensor 444 is sent to the control unit 150, such that the control unit 150 can use the information from the air pressure sensor 444 to determine operation of the air system 404. For example, the control unit 150 may determine if the air pump 408 or the relief valve 410 need to be active or not active. The control unit 150 may be communicatively coupled to both the air pump 408 and the relief valve 410 to turn either on or off depending on the determination. For example, the air pressure sensor 444 can detect a current air pressure of the bag and the control unit 150 can determine (e.g., based on a user input, additional sensors, etc.) that the current air pressure is too low and turn on the air pump 408 such that more air can enter the air bag 406 to increase the current air pressure.

As shown in FIG. 4, the water system 412 can include a water pressure sensor 446 that is communicatively coupled to the control unit 150. The water pressure sensor 446 may be coupled to the water bag 414. The water pressure sensor 446 is configured to detect a water pressure of the water bag 414 and/or a change in the water pressure of the water bag 414. As described herein, the water pressure of the water bag 414 changes as the water pump 416 and the relief system 432 add and remove water from the water bag 414, respectively. Information received from the water pressure sensor 446 is sent to the control unit 150 and the control unit 150 uses the information to determine if the water pump 416 or the relief system 432 need to be turned on or off. The control unit 150 is communicatively coupled to the water pump 416 and the relief system 432 to turn either on or off.

The water system 412 can include a water level sensor 448 coupled to the water tank system 418 and communicatively coupled to the control unit 150. The water level sensor 448 is configured to detect a water level of the water tank system 418 (e.g., the hot water tank 420, the cold water tank 422) to determine if the water level is low. In various embodiments, the water level sensor 448 may be or include a mechanical lever to determine and detect a low water level. Information from the water level sensor 448 is sent to the control unit 150. The control unit 150 can use the information from the water level sensor 448 to determine if water needs to be added to the water tank system 418. In various embodiments, as described herein, an alert may be sent to the user when the water level of the water tank system 418 is low. The water tank system 418 includes a tank filler connector 456 for the hot water tank 420 and the cold water tank 422 such that water can enter the hot water tank 420 and the cold water tank 422 by the tank filler connector 456 when the water tank system 418 is low.

The water system 412 can include at least one sensor to determine a temperature of the water in the water tank system 418. For example, the at least one sensor may be a water temperature sensor (e.g., a hot water sensor 450 and a cold water sensor 452). The hot water sensor 450 and the flow meter 454 can be coupled to the hot water tank 420 and the cold water tank 422, respectively. The hot water sensor 450 and the cold water sensor 452 are configured to detect (e.g., determine) a water temperature of the water in the water tank system 418 and communicate information on the water temperature to the control unit 150. The control unit 150 can use the information related to the water temperature to determine if a heating/cooling system (e.g., a heat pump, the Peltier assembly 470) of the water system 412 needs to be adjusted. Additionally, a user can set (e.g., via a user input) a desired temperature for the lounger 100 (e.g., a portion of the lounger 100) and the control unit 150 can use the desired temperature to adjust the heating/cooling system such that the water circulated to the user is at the desired temperature. In various embodiments, the water system 412 can include a water temperature sensor coupled to the water bag 414 to determine the water temperature of water in the water bag 414.

The compression system 442 can include a flow meter 454 that is communicatively coupled to the control unit 150. A portion of the water from the water bag 414 can be drained into the flow meter 454 and released. The flow meter 454 and the control unit 150 may use the portion of the water to determine flow rate, volume of water, water pressure, etc. of the water. The determined information may be used to further monitor usage of the water and regulate flow of the water throughout the lounger 100.

As shown in FIG. 15, the lounger 100 can include a user interface 160 that is communicatively coupled to the control unit 150. In various embodiments, the user interface 160 includes buttons, switched, etc. to receive an input from a user. For example, the user interface 160 can include an ON/OFF button, switch, etc. for a user to control the lounger 100. The user interface 160 can include a display configured to display information on the lounger 100. The display may display information relating to malfunctioning error, water levels of the water system 412 (e.g., as determined by the water level sensor 448), etc.

In various embodiments, the lounger 100 may be remotely controlled with a user device 162. The user device 162 is communicably connected to the lounger 100 via the control unit 150 and is configured to allow a user to remotely adjust the lounger 100. For example, the user can use the user device 162 to adjust where water is being circulated (e.g., only directing water to an arm sleeve 106a, directing water to both the body seat 104 and a leg sleeve 106b, etc.). As described herein, the configuration of the lounger 100 and the layers of the lounger sections 103 allows for selective hydrotherapeutic outputs to be delivered throughout the lounger 100. As a result, one area (e.g., lounger section 103) of the lounger 100 may have a water temperature and/or a pressure delivered that is different from a water temperature and/or a pressure delivered to another area (e.g., lounger section 103) of the lounger 100. The user device 162 can be a smartphone, remote, screen, etc. Additionally, the user device 162 can be an application installed onto a smartphone. In various embodiments, the user device 162 is coupled to the lounger 100 via a physical electronic component (e.g., a wire).

The lounger 100 can include a power supply 170 that is configured to provide power to electrical components of the lounger 100. For example, the power supply 170 can power the control unit 150 and the user interface 160. The power supply 170 may be a battery system or a power system configured to receive electrical energy from an external power source. In various embodiments, the heating/cooling system (e.g., the Peltier assembly 470) is powered by the power supply 170 to heat and cool the water tank system 418.

In various embodiments, as shown in FIG. 15, the lounger 100 includes a plurality of detection sensors l20 (e.g., on the body seat 104, on the sleeve 106). The detection sensors 120 are communicatively coupled to the control unit 150. The detection sensors 120 are configured to sense areas of the user as the user contacts the lounger 100 and receive data related to the areas of the user. The detection sensors 120 may be on a portion of the lounger 100 that directly engages with the user. For example, the detection sensors 120 may be on the water bag 414 or the air bag 406 of the lounger section 103. The data received may be related to temperature, pressure, or movement, among others. The detection sensors 120 can be an electromyography sensor, a thermal sensor, a force sensor, a pressure sensor, any biochemical sensor, or any combination thereof, among others. The detection sensors 120 are configured to receive information related to the user and send the information to the control unit 150 to determine at least one of inflammation, muscle pain, tissue injuries, swelling, or strain (e.g., muscle strain), among other muscle and body conditions. For example, the detection sensors 120 can be a thermal sensor. In such example, the thermal sensor may determine areas of increased temperature by measuring a skin temperature of the user to determine areas of inflammation of the user.

The control unit 150 can receive information from the detection sensors 120 to determine areas of the user in which hydrotherapeutic outputs from the lounger 100 may be necessary. The control unit 150 can then control operation of the lounger 100 (e.g., the lounger sections 103, the body seat 104, the sleeves 106) based on the received information. For example, the control unit 150 can send a signal to at least one of the lounger sections 103 to apply pressure to the user at the determined areas. For example, the detection sensors 120 and the control unit 150 can determine that an arm of the user has muscle pain and the control unit 150 can control the water system 412 to add water, by the water pump 416, to the water bag 414 of the arm sleeve 106. As such, the lounger 100 can provide specific and targeted therapy to the user. Additionally, dependent on the need of the user, hot water can be circulated around a first area of the lounger 100 and cold water can be circulated around a second area of the lounger 100. For example, hot water can be circulated around an arm sleeve 106a and cold water can be circulated around a leg sleeve 106b simultaneously.

A user of the lounger 100 can use the user interface 160 and/or the user device 162 to identify parts of the lounger 100 that they want water and/or pressure to circulate through. The control unit 150 can then use the inputs from the user to activate the air system 404 and/or the water system 412 to provide hydrotherapy. Additionally, a user can identify parts of a body of a user that they want targeted and the control unit 150 can determine a corresponding area of the lounger 100 to circulate water and/or pressure to. In various embodiments, the user interface 160 may include presets related to different hydrotherapeutic outputs. In such embodiments, the control unit 150 is configured to store the presets and/or information related to the presets. The presets may include a sequence of controls for each of the lounger sections 103 (e.g., water temperature, water pressure, pressure, etc.). The presets may include presets for different areas of the body, targeted conditions (e.g., inflammation, muscle pain, tissue injuries, etc.), and/or level of intensity of the hydrotherapeutic outputs. In various embodiments, the user may create at least one preset.

Advantageously, the lounger 100 allows for a user to receive hydrotherapy in various body parts of the user. The configuration of the lounger 100 and the water system 412 provides users with both hot therapy and cold therapy. Additionally, the lounger 100 allows for the users to target specific parts of a body of the user by the user interface 160 and by the power supply 170 and the hydrotherapy received can be easily adjustable dependent on the user and specific needs of the user.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

The terms “coupled,” “connected,” and the like, as used herein, mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

The construction and arrangement of the elements of the assembly as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements, the position of elements may be reversed or otherwise varied, and the nature or number of discrete elements or positions may be altered or varied.

Additionally, the word “exemplary” is used to mean serving as an example, instance, or illustration. Any embodiment or design described herein as “exemplary” is not necessarily to be construed as preferred or advantageous over other embodiments or designs (and such term is not intended to connote that such embodiments are necessarily extraordinary or superlative examples). Rather, use of the word “exemplary” is intended to present concepts in a concrete manner. Accordingly, all such modifications are intended to be included within the scope of the present disclosure. Other substitutions, modifications, changes, and omissions may be made in the design, operating conditions, and arrangement of the preferred and other exemplary embodiments without departing from the scope of the appended claims.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Other substitutions, modifications, changes and omissions may also be made in the design, operating conditions and arrangement of the various exemplary embodiments without departing from the scope of the present invention. For example, any element disclosed in one embodiment may be incorporated or utilized with any other embodiment disclosed herein. Also, for example, the order or sequence of any process or method steps may be varied or re-sequenced according to alternative embodiments. Any means-plus-function clause is intended to cover the structures described herein as performing the recited function and not only structural equivalents but also equivalent structures. Other substitutions, modifications, changes and omissions may be made in the design, operating configuration, and arrangement of the preferred and other exemplary embodiments without departing from the scope of the appended claims.

What is claimed is:

1. A lounger for hydrotherapy, comprising:
a body seat;
a plurality of sleeves, at least one of the sleeves coupled to the body seat;
the at the least one of the sleeves comprising:
an air system including an air pump and an air bag, the air pump configured to deliver air to the air bag, and
a water system including a water pump and a water bag, the water pump configured to deliver water to the water bag; and
a control unit communicably coupled to the air system and the water system to control operation of the lounger.

2. The lounger of claim 1, wherein the control unit is configured to control at least one of a pressure of air delivered to the air bag or a water temperature of water delivered to the water bag.

3. The lounger of claim 1, wherein the air system includes a relief valve coupled to the air bag, the relief valve configured to release air from the air bag.

4. The lounger of claim 1, wherein the water pump is connected to a water tank system, the water tank system including at least one tank configured to contain the water.

5. The lounger of claim 4, wherein the at least one tank includes a hot water tank to contain water at a first temperature and a cold water tank to contain water at a second temperature, the first temperature higher than the second temperature.

6. The lounger of claim 5, wherein the water system comprises a three-way diverter valve connected to the hot water tank, the cold water tank, and the water pump, the three-way diverter valve to selectively adjust between water from the cold water tank to transport to the water pump and water from the hot water tank to transport to the water pump.

7. The lounger of claim 4, wherein the water system includes a relief system to release water from the water bag into the water tank system.

8. The lounger of claim 4, wherein the water system includes a Peltier assembly coupled to the at least one tank to control a water temperature of the water in the at least one tank.

9. The lounger of claim 1, further comprising a plurality of detection sensors communicatively coupled to the control unit, the control unit configured to receive information from the plurality of detection sensors to determine operation of the air system and the water system of the at least one of the plurality of sleeves.

10. The lounger of claim 9, wherein the plurality of detection sensors are configured to determine at least one of inflammation, muscle pain, tissue injury, swelling, or strain.

11. The lounger of claim 1, wherein at least one of the plurality of sleeves has a shape conforming to a portion of a body shape of a human.

12. The lounger of claim 1, wherein:
the air system comprises an air pressure sensor communicatively coupled to the control unit, the air pressure sensor configured to detect air pressure of the air bag; and
the water system comprises a water temperature sensor communicatively coupled to the control unit, the water temperature sensor configured to determine water temperature of water in the water system.

13. A lounger for hydrotherapy, comprising:
a base; and
a lounger section coupled to the base, the lounger section comprising:
an air system comprising an air bag, the air bag configured to receive air at a pressure; and
a water system comprising a water bag coupled to the air bag, the water bag configured to receive water at a temperature;
wherein the lounger section is configured to fit at least partially around a part of a user to deliver a hydrotherapeutic output from the air bag and the water bag to the part of the user.

14. The lounger of claim 13, wherein the lounger section is an arm sleeve, a leg sleeve, or a body seat.

15. The lounger of claim 13, further comprising a control unit communicably coupled to the air system and the water system to control at least one of the pressure of air received by the air bag or the temperature of water received by the water bag.

16. The lounger of claim 15, wherein the control unit is configured to store at least one preset, the at least one preset including a sequence of controls for the air system and the water system.

17. The lounger of claim 15, wherein:
the lounger further comprises a detection sensor positioned on the lounger section and communicatively coupled to the control unit, the detection sensor configured to receive data related to the part of the user; and
the control unit is configured to receive data from the detection sensor to determine operation of the air system and the water system.

18. The lounger of claim 13, further comprising a top cover coupled to the lounger section, the top cover and the lounger section configured to form an enclosure, the top cover comprising the air system and the water system.

19. The lounger of claim 13. wherein a first end of the air bag is configured to couple to a second end of the air bag to form an enclosure.

20. A lounger section for a lounger, comprising:
a frame support coupled to a base of the lounger;
an air system coupled to the frame support, the air system comprising an air bag configured to contain air at a pressure, an air pump coupled to the air bag and configured to deliver air to the air bag, and an air pressure sensor configured to detect the pressure of air; and
a water system coupled to the air system, the water system comprising a water bag configured to contain water at a temperature, a water pump coupled to the water bag and configured to deliver water to the water bag, and a water temperature sensor configured to detect the temperature of water, the water bag coupled to the air bag;
wherein the water temperature sensor and the air pressure sensor are communicably coupled to a control unit of the lounger.
